(19) United States
(12) Patent Application Publication
(10) Pub. No.: US 2023/0002971 A1
(43) Pub. Date: Jan. 5, 2023

(54) METHOD AND PLANT FOR OBTAINING CELLULOSE FIBRES

(71) Applicant: LEINICH BETEILIGUNGEN GMBH, Graz (AT)

(72) Inventors: Hermann DAUSER, Freilassing (DE); Felix NUSSBAUMER, Seekirchen (AT); Helge LEINICH, Graz (AT)

(73) Assignee: LEINICH BETEILIGUNGEN GMBH, Graz (AT)

(21) Appl. No.: 17/779,837

(22) PCT Filed: Nov. 27, 2020
(86) PCT No.: PCT/AT2020/060421

§ 371 Date: May 25, 2022

(30) Foreign Application Priority Data
Nov. 29, 2019 (AT) A 60265/2019
Apr. 24, 2020 (AT) A 60114/2020

(51) Int. Cl. D21C 1/02; D21C 3/00; C12M 1/00; C12M 1/107

(52) U.S. Cl. CPC D21C 1/02; D21C 3/003; C12M 45/06; C12M 21/04; C12M 45/20

(57) ABSTRACT

The invention relates to a method for obtaining cellulose fibres from fibrous biomass, in which: the biomass is first subjected to thermo-pressure hydrolysis, preferably with steam explosion, in a thermo-pressure hydrolysis plant, and then separation of the fibrous sludge obtained from the thermo-pressure hydrolysis plant is carried out in at least one separation plant, wherein a press cake of cellulose fibres, preferably with a dry material content of over 20%, preferably of over 25%, and a filtrate of flowable, solids-rich thin sludge are obtained, and wherein the thin sludge is fed to a biogas plant as a fermentation substrate to obtain biogas. The invention also relates to a plant for carrying out this method.

The invention relates to a method for obtaining cellulose fibres from fibrous biomass, and to an associated plant.

In order to produce pulp as a main component of paper products, depending on the origin and location, wood that has grown for at least 7 years for example in tropical wood plantations using fertilizers, herbicides, pesticides, fungicides and formicides (tropical pulp production) or wood that has grown in natural forests for between 60 and 120 years (pulp production in temperate zones) is harvested and stripped of branches by suitable harvesting machines, thereby using a considerable amount of energy.

The logs that have been cut to length and partially already debarked are then transported to pulp mills, which are usually up to 250 km away.

In modern pulp mills, usually around 2.5 tonnes of wood are required in order to produce one tonne of pulp. Using a considerable amount of energy, the material, which is usually in the form of logs, is chopped into wood chips. The wood chips are transported from the woodyard to a tank, in which they are typically impregnated with steam and alkali for further processing.

Following the impregnation, the wood chips are usually transferred to a continuous digester. In this digester, the lignin is dissolved by means of pressure, temperature and white liquor (sodium hydroxide and sodium sulphide) in a chemical/thermal pulping process in order to expose the pulp fibres. The raw fibre obtained then exists as unbleached, not yet sufficiently finely shredded pulp, which is then subjected to various cleaning and washing steps in order to free it from impurities. The resulting wash liquor (also called black liquor) represents a significant environmental burden that requires complex technical measures in effluent treatment, including the incineration of thickened liquor.

Besides undissolved and/or mineral components such as phosphates and silicates, these impurities to be removed also include, in particular, organic substances such as hemicellulose, which is present as dissolved sugar, and lignin. In the course of conventional effluent treatment, the organic substances are mineralized, and mineral substances are converted into non-reactive, harmless substances. The cleaned effluent is then discharged into bodies of water, and organic residues are burned.

The unbleached pulp can be bleached in different bleaching processes, most of which are nowadays chlorine-free. Depending on grade requirements, the finished pulp is transported directly to paper machines for integrated paper production or is dried in web dryers or flash dryers in order to be made transportable as bales or rolls.

This established method for producing pulp therefore requires a high input of expensive, slow-growing raw materials, chemicals and energy.

The object of the invention is therefore to provide an alternative method for producing pulp, which is environmentally friendly, sustainable, energy-saving and at the same time economical.

This object is achieved according to the invention by a method of the type mentioned in the introduction in that the fibres of the biomass are first subjected to thermo-pressure hydrolysis, preferably with steam explosion, in a thermo-pressure hydrolysis plant, and then a separation of the fibrous sludge obtained from the thermo-pressure hydrolysis plant takes place in at least one separation plant, wherein a press cake formed of cellulose fibres, preferably having a dry matter content of more than 20%, and a filtrate formed of a flowable, high-solids, thin sludge are obtained, and wherein the thin sludge is fed to a biogas plant as a fermentation substrate in order to obtain biogas.

According to the invention, it is preferably provided that the fibrous biomass is first pulped by means of thermo-pressure hydrolysis with steam explosion. The pulp fibres are exposed during this method step, in a manner analogous to digesting the wood chips with white liquor and then with black liquor according to the prior art. Thermo-pressure hydrolysis with subsequent steam explosion has already proven itself in the production of fermentation substrates from energy crops, wherein these fermentation substrates are then converted into biogas by anaerobic fermentation in a biogas plant. One such method can be found, for example, in EP 2 177 280 B1.

The generation of biogas from plant biomass by anaerobic fermentation is an established technology. The raw materials used for this are mainly so-called energy crops, usually in the form of silage. These raw materials contain different proportions of fibrous materials consisting of lignocellulose bonds, which are difficult to break down in a biogas plant. The residues from the fermentation therefore still contain large proportions of stable fibrous materials, which after being discharged from the fermentation process are disposed of without being used for energy.

The greater the proportion of these stable fibres in the biomass, the lower the success and thus the economic efficiency of the fermentation. As a result, most biogas plants use only crops that have a relatively low fibre content, such as maize, but the intensive cultivation of these crops is expensive and is not without controversy from an ecological standpoint. In general, biogas plants based on energy crops are under increasing pressure, in particular because the costs of producing the preferred raw materials are increasing and the revenues based on state-subsidized tariffs are time-limited or are even degressive in some models.

Under these circumstances, it is difficult for the operators of biogas plants to use alternative biomass sources which are also more environmentally friendly, since these usually have lower yields per hectare and at the same time higher fibre contents than the usual silage from energy crops.

The use of suitable technologies, in particular thermo-pressure hydrolysis with steam explosion, makes it possible for biogas plants to use woodier, i.e. lignocellulose-containing, raw materials as an alternative to energy crops since, after being treated by thermo-pressure hydrolysis, these can be fermented and converted into biogas with a high degree of efficiency.

However, this superior technology is to date established only in individual cases in biogas plant technology because it entails high investment and increased operating costs. Against the background of expiring subsidized feed-in tariffs and a lack of other incentives, there is a need for optimized processes with higher added value.

Studies by the applicant have now shown that the known method of thermo-pressure hydrolysis lends itself as a first method step in the production of pulp, wherein according to the invention the fibrous sludge obtained in this first method step is still mechanically separated into cellulose fibres and filtrate in the form of thin sludge. The method according to the invention therefore makes it possible to produce a pulp from fibre-rich biomass without using environmentally harmful chemicals and with lower energy consumption, wherein the biomass can be selected from a large number of different plant materials. In the context of this disclosure, the term “pulp” will be understood to mean a fibre cake obtained from biomass by thermo-pressure hydrolysis and cleaned, wherein the biomass used may be not only wood, but also any suitable crops or crop residues.

At the same time, a fermentation substrate is produced which is suitable for generating energy in a biogas plant. The impurities separated in the form of thin sludge from the fibres in the method according to the invention contain the bulk of the proportion of biomass that can be used for energy in a biogas plant. Studies have revealed a proportion of more than 60% of the usable energy potential. In order to avoid long transport routes, it is particularly preferred that this biogas plant is located in the immediate vicinity of the pulp production plant, wherein the biogas obtained in the biogas plant is advantageously used as an energy source for the method according to the invention.

After suitable conditioning (shredding, ensiling, etc.), the biomass, which is preferably produced regionally as a field crop or by-product, is first subjected to a pre-treatment at elevated pressure and elevated temperature (thermo-pressure hydrolysis, preferably with steam explosion), namely on site or in the immediate vicinity of a biogas plant. Immediately thereafter, the treated product is separated into a processed fibre fraction (cellulose), which is used as a raw material for paper production, and a highly contaminated sub-stream, which is used as a fermentation substrate in the biogas plant.

Fermentation residues occur as a residual product of biogas production and contain, in addition to mineral and organic residues of the fermented substances, also mineral fertilizer components (nitrogen, phosphorus, potassium, trace elements) and a high concentration of lignin, which is inert in the fermentation process. As part of sustainable agriculture, these nutrient-rich fermentation residues from the biogas plant are returned as fertilizing agents to the areas being cultivated for the plant-based raw materials, thereby also achieving, in particular, an improvement in the humus balance.

By combining the described fibre processing with a biogas plant, a number of advantageous effects are achieved:
Since the fibre pulping is not carried out in a paper mill, where the thin sludge that occurs during the subsequent separation has to be disposed of or treated as effluent, the ecological balance of paper production is significantly improved.
In addition, the transport costs are significantly reduced due to the regional approach to extracting raw materials, in which raw material cultivation and pulp production take place locally, and due to the fact that the finished product (compressed pulp fibres), instead of the considerably more voluminous raw material (biomass), is transported for example to a paper mill that is situated as close as possible.
The use of regionally obtained pulp also reduces the use of pulp imported from overseas, which is produced for example from plantation-grown wood.
By returning lignin to the agricultural cultivation areas in a targeted manner, the fertility of the soil is maintained or even improved due to the effect thereof on the humus, so that intensive and yet sustainable agricultural production of raw materials is possible.
By returning to the agricultural land the silicates that have dissolved out of the plant structure, which are likewise contained in the fermentation substrate produced and thus also in the fermentation residue, a substance that acts as a nutrient store is added to the soil, which improves the soil quality over the long term.
By returning the mineral fertilizing substances nitrogen, phosphate, potassium and trace elements, which have likewise dissolved out of the plant-based raw material and are contained in the fermentation substrate, this reduces the need for artificial fertilizers in raw material production.
The use of the thermo-pressure treatment plant as a pre-treatment for the biomass in the biogas plant permits the use of alternative raw materials which contain more fibres, the production or extraction of which may be ecologically more sustainable than that of the usual energy crops, as well as the use of by-products such as unused straw or harvested residual plants of various field crops (so-called co-products).

It should also be noted that the production of pulp from grasses and other fast-growing plants cultivated in fields can bind significantly larger amounts of carbon dioxide than biomass production from wood, for example in plantation economy, and can thus make a significant positive contribution to climate protection.

For the value chain of a biogas plant, the economically separate use of the pulp fibres opens up the possibility of generating additional income in addition to generating energy, for example by selling the pulp fibres to the paper industry. These fibres, which are difficult to convert into biogas, have until now largely been output as residue (solid fermentation residue).

In order to obtain additional cleaning and thus an improvement in the quality of the pulp, it is provided in one particularly preferred embodiment of the invention that the fibrous sludge obtained after the thermo-pressure hydrolysis is adjusted in a first mashing tank to a dry matter content of preferably 3% to 20%, and then the separation of the fibrous sludge takes place in at least one separation plant. Due to the intermediate step of mashing the fibrous sludge obtained from the thermo-pressure hydrolysis in a mashing tank, a value for the dry matter content that is optimal for the subsequent separation is obtained.

In order to obtain finer pulp, it is provided in a further embodiment of the method according to the invention that, before the mashed fibrous sludge from the mashing tank is separated, a fibre separation or singulation of fibre bundles takes place in at least one disintegrator, and then the separation takes place in the first separation plant. For this purpose, the dry matter content of the fibrous sludge is preferably adjusted to 3% to 10% before the latter is fed to the disintegrator.

In an alternative embodiment of the invention, it is provided that, after the fibrous sludge has been separated in a first separation plant, the press cake obtained is fed to a mashing tank in order to set a dry matter content of preferably 3% to 20%, particularly preferably 3% to 10%, and then the fibrous sludge is fed to at least one disintegrator in order to obtain a fibre separation of the fibre bundles contained in the fibrous sludge, and thereafter a separation of the fibrous sludge takes place in at least one further separation plant.

In the fibrous sludge drawn off from the thermo-pressure hydrolysis plant, the desired pulp is present in the form of fibre bundles which are bonded to one another by natural polymers, in particular lignin and the like. By mashing the fibrous sludge in the mashing tank, a first dissolving-out of undesired components already takes place, as well as the physical separating-out of any insoluble components by sedimentation. At the same time, adjusting the dry matter content to 3% to 10% permits an improved fibre separation in the at least one disintegrator.

Depending on the type of biomass used, it may be necessary for the fibrous sludge to pass through the at least one disintegrator multiple times. In this case, preferably the fibrous sludge is mashed again in the mashing tank, and the fibre separation in the disintegrator is repeated at least once, preferably multiple times, in a cyclic process between the mashing tank and the disintegrator. As an alternative or in addition, it may be provided that additional fibrous sludge, which has not yet been treated in the disintegrator, is added to the material located in the mashing tank.

Depending on the desired quality and properties of the end product, fibre shredding may be provided in addition to or as an alternative to fibre separation.

Besides a high pulp quality, the method described above using at least one, preferably two or more separation plants makes it possible to obtain thin sludge as a waste product of pulp production, wherein the filtrate is at least in part fed to a biogas plant as a fermentation substrate.

It is particularly preferably provided that the filtrate from the separation plants, which is in the form of a thin sludge, is at least in part returned to the process. In this case, it is particularly preferably fed to the mashing tank in order to adjust the dry matter content of the fibrous sludge. As an alternative or in addition, the filtrate may also be added directly to the fibrous sludge before the latter is conveyed into a separation plant.

The thin sludge fed to the biogas plant as a fermentation substrate may be thickened, preferably by filtration (for example fine filtration, microfiltration or ultrafiltration) in order to reduce the volume flow. The resulting filtrate, a sub-stream having a lower solids content, is advantageously fed into the method according to the invention as mashing water for the thermo-pressure hydrolysis plant and/or elsewhere, thereby further reducing the water consumption in the method according to the invention.

In one particularly preferred embodiment of the invention, it is provided that the thin sludge is collected in two sub-fractions, wherein a first sub-fraction having a lower solids content is returned to the process, while a higher-solids fraction is fed to the biogas plant as a fermentation substrate. These different fractions are withdrawn for example from different areas of the at least one separation plant and are preferably collected in separate collection tanks.

In order to be better able to store and transport the pulp produced by the method according to the invention, it may be provided that, prior to being stored as an end product, the press cake obtained from the at least one separation plant is subjected to a stabilization step, in particular by adding preserving chemicals, and/or to a heat treatment.

In order to further improve the quality of the end product, it is provided in a further variant of the invention that the press cake obtained from the at least one separation plant is subjected to a further cleaning step in a mixing reactor, wherein the wash water is separated from the cleaned fibre cake in a further separation plant. The mechanically treated and dewatered fibres are thus subjected to a further, additional washing step, wherein the wash water used here is advantageously clean water that is free of contaminants. It is particularly advantageous if the wash water is added to the press cake obtained from the previous separation step, for example in a ratio of fibrous sludge to wash water of 1:1 to 1:2. After sufficient contact with the wash water, the cleaned fibre is subjected to a final dewatering step in order to restore the desired solids content in the end product.

The slightly contaminated wash water obtained after this cleaning step is preferably returned to the process according to the invention, wherein it is particularly preferably provided that said wash water is added to dry biomass, requiring the addition of water, in order then to be able to process said biomass in the thermo-pressure hydrolysis plant. This results in a water cycle that is particularly advantageous both in terms of the method and ecologically.

One significant advantage of the method according to the invention lies in particular in that a large number of fibrous materials in the form of plant biomass can be used. Energy crops such as maize, Silphium perfoliatum, and/or harvest residues with a sufficient cellulose or lignocellulose content have proven to be particularly suitable here, as well as by-products such as straw and/or green cuttings. Regional raw materials and/or residues such as harvest by-products or green cuttings can therefore be used to obtain pulp while at the same time generating energy in the form of biogas. It is particularly preferably provided that the biogas obtained in the biogas plant is used as an energy source in the method according to the invention, in particular for the thermo-pressure hydrolysis plant.

At the same time, it is particularly preferably provided that the non-recyclable residues occurring in the biogas plant are used as fertilizing agents in agriculture. Besides the usable organic components, the fermentation substrate obtained in the method according to the invention contains in particular lignin and silicates, which cannot be converted in the biogas plant. However, these residues from the biogas plant can significantly improve the condition of the soil. For instance, lignin forms an important basic building block for the formation of humus, while silicates act as a mineral adsorbent that significantly influences the nutrient balance of the soil.

The object mentioned above is further achieved by a plant according to the invention in that a thermo-pressure hydrolysis plant is provided for subjecting the fibres of the biomass firstly to thermo-pressure hydrolysis with steam explosion, wherein the thermo-pressure hydrolysis plant is connected via at least one feed line to at least one first separation plant, preferably a screw press, into which the fibrous sludge drawn off from the thermo-pressure hydrolysis plant can be fed by means of at least one conveying device, preferably a screw conveyor and/or a thick-matter pump, wherein the filtrate obtained from the first separation plant in the form of a flowable, high-solids, thin sludge can be fed to a biogas plant via at least one further feed line.

An improved separation of the fibrous sludge into pulp fibres and filtrate in the form of thin sludge is obtained if additionally a mashing tank is provided, which is arranged between the thermo-pressure hydrolysis plant and the first separation plant.

Particularly in the case of biomass having a high lignin content, the pulp fibres are in the form of bonded pulp bundles after the thermo-pressure hydrolysis with steam explosion, which impairs the efficiency of the subsequent separation step and consequently the quality of the pulp. It is therefore particularly preferably provided that the mashing tank is connected to at least one disintegrator, wherein the at least one disintegrator is connected to the first separation plant preferably via storage tanks, in which the singulated cellulose fibres can be intermediately stored.

It may alternatively be provided that the mashing tank is arranged downstream of the at least one first separation plant, wherein preferably the mashing tank is connected to the at least one disintegrator, and wherein the at least one disintegrator is connected to at least one further separation plant preferably via at least one storage tank.

For easier processing and further use, the filtrate obtained from the first separation plant and/or second separation plant is collected in at least one collection tank, wherein preferably the at least one collection tank is connected to the mashing tank via at least one recirculation line. Furthermore, the at least one collection tank is connected to the biogas plant via at least one further feed line.

The invention will be explained in greater detail below on the basis of non-limiting exemplary embodiments together with associated figures, in which:

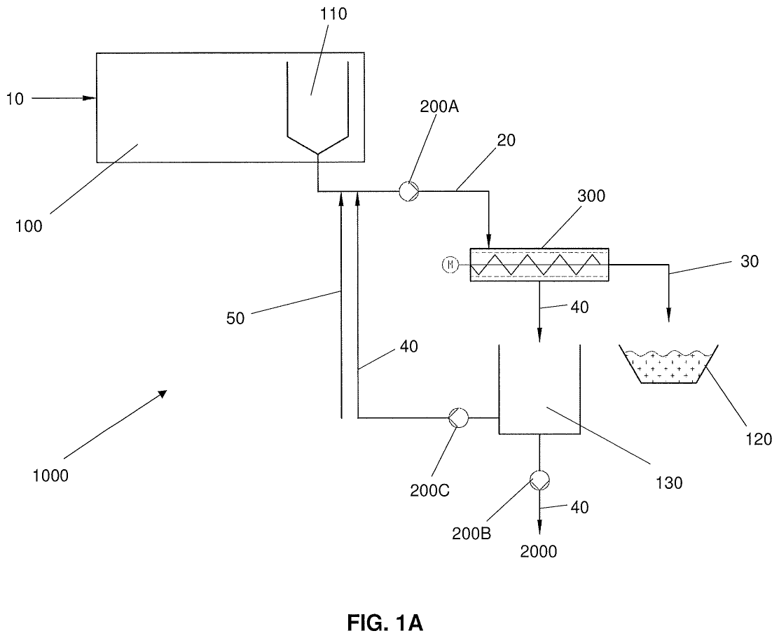

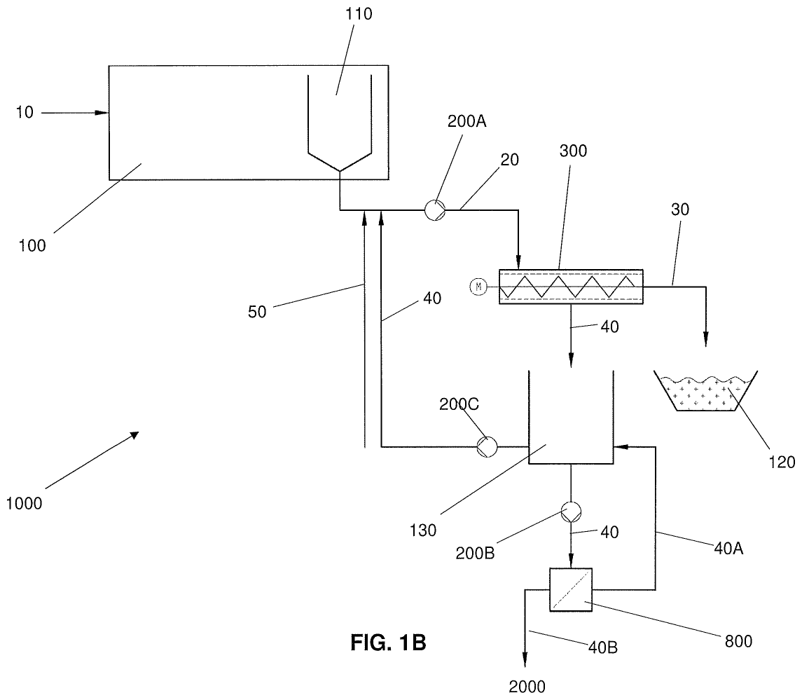

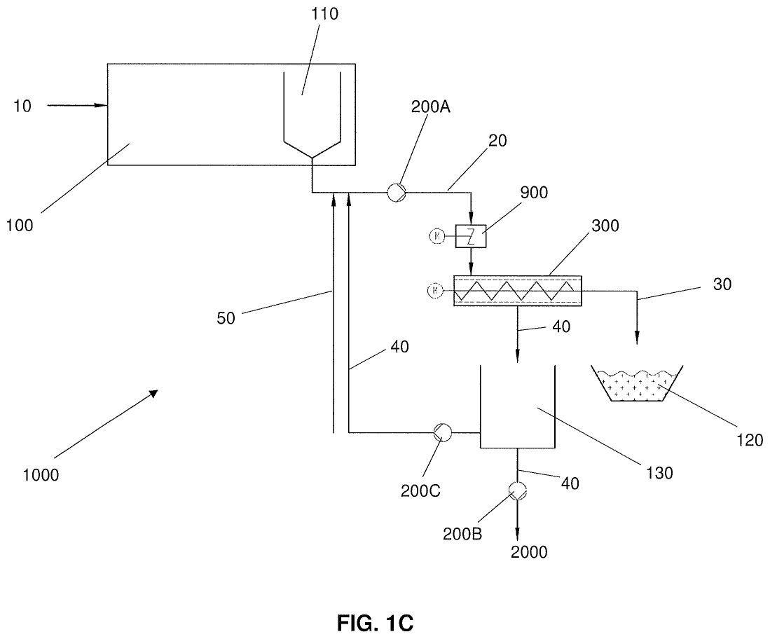

FIG. 1A shows a schematic illustration of a first embodiment variant of the plant according to the invention,

FIG. 1B shows a variant of the plant from FIG. 1A,

FIG. 1C shows a further variant of the plant from FIG. 1A,

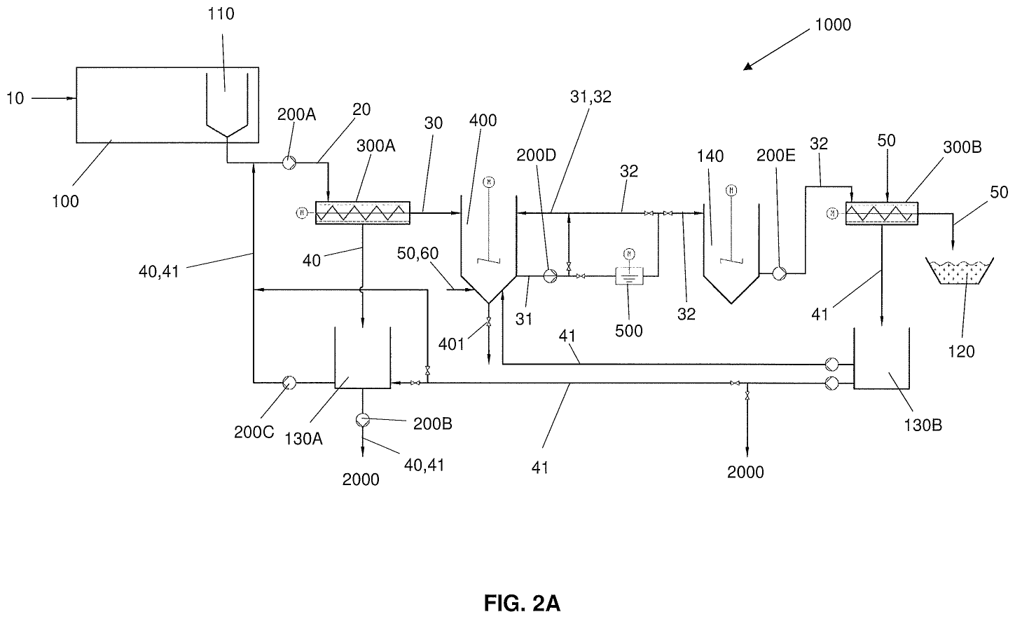

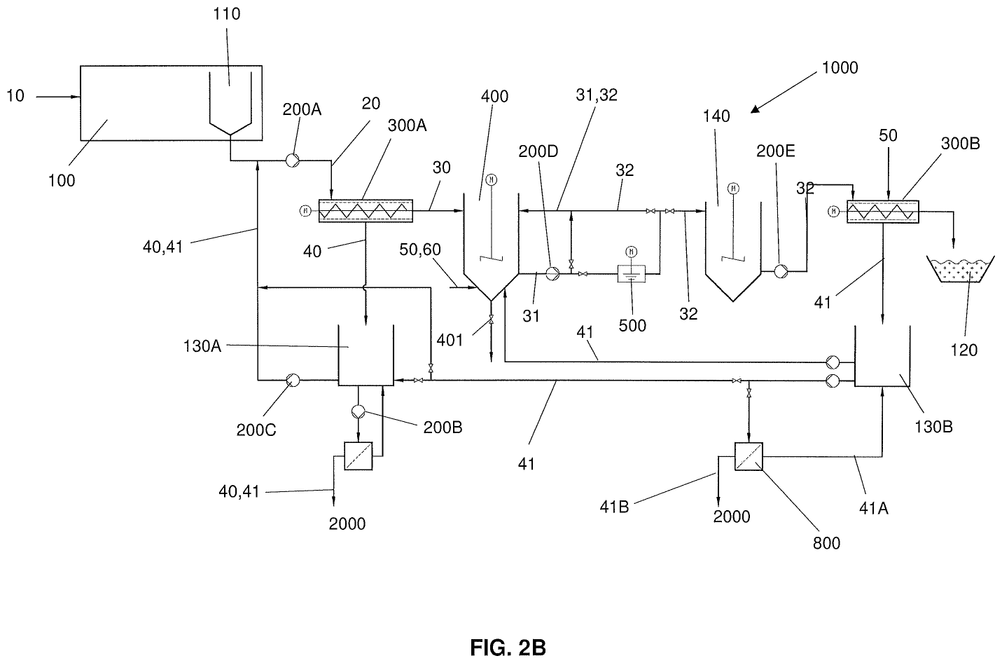

FIG. 2A shows a schematic illustration of a second embodiment variant of the plant according to the invention,

FIG. 2B shows a variant of the plant from FIG. 2A,

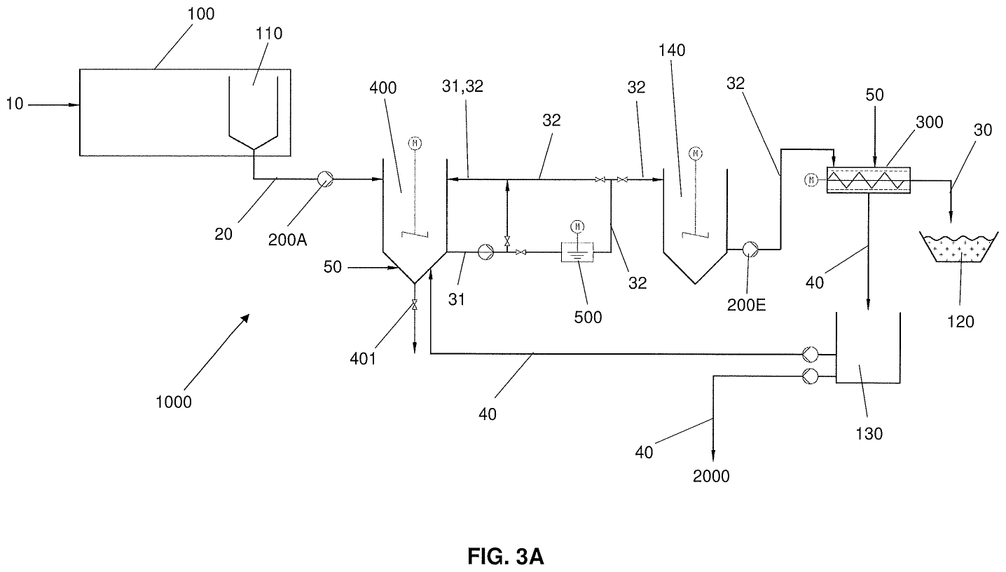

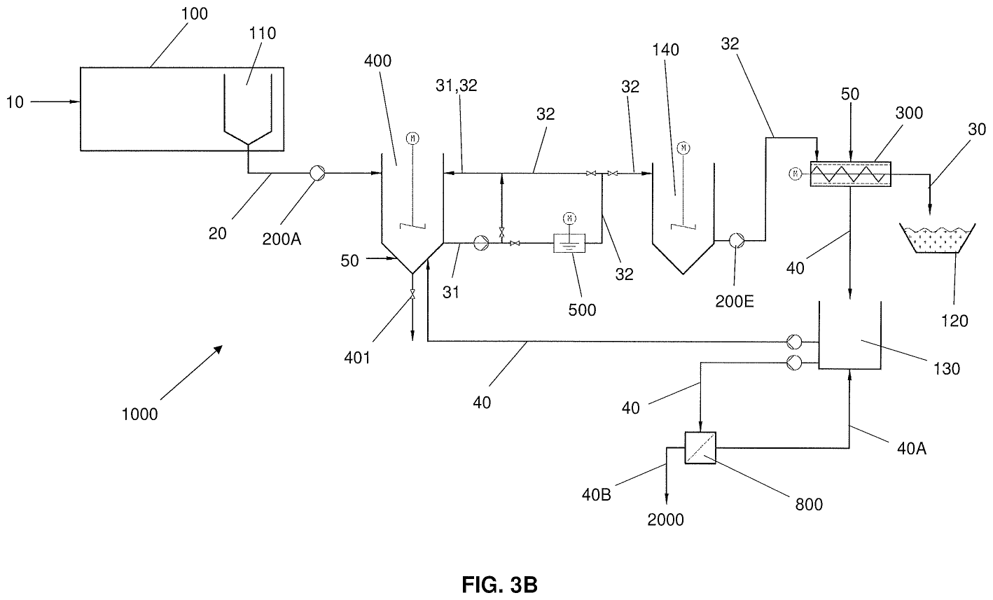

FIG. 3A shows a schematic illustration of a third embodiment variant of the plant according to the invention,

FIG. 3B shows a variant of the plant from FIG. 3A,

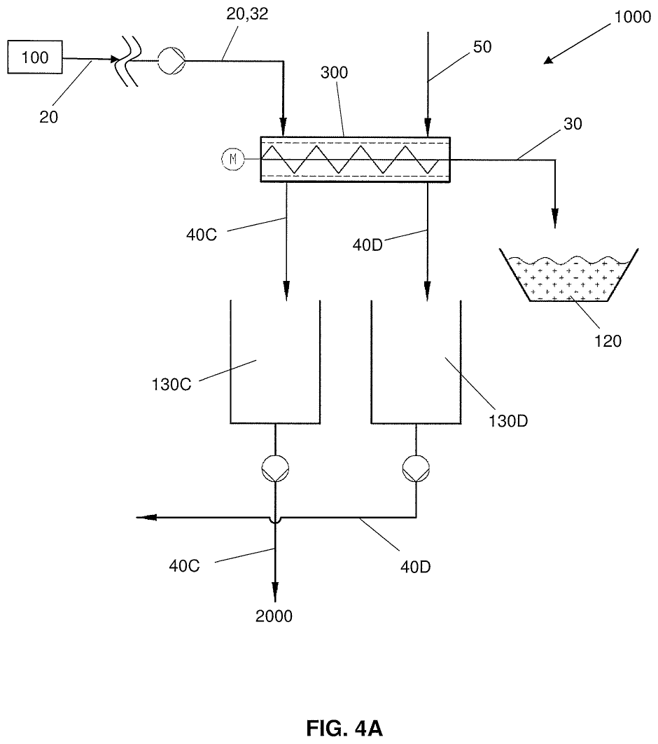

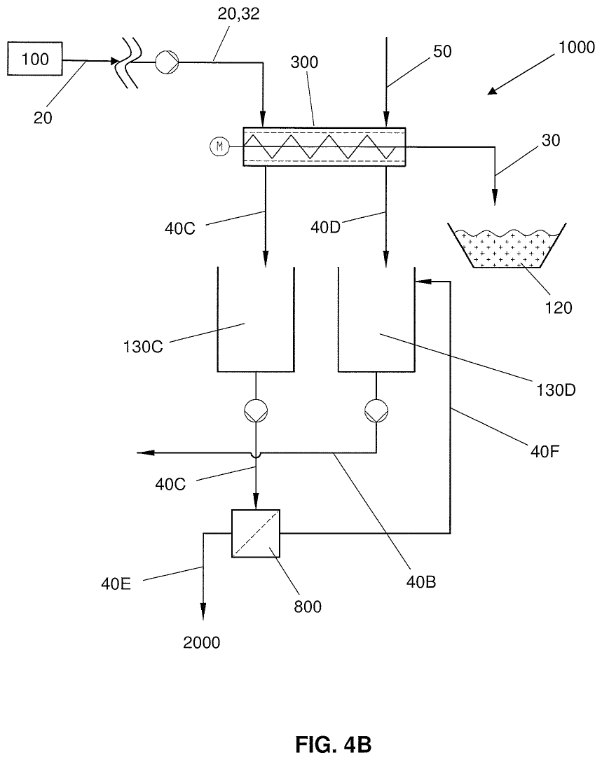

FIG. 4 shows a schematic detail view of one particular embodiment of the second separation plant from FIG. 2,

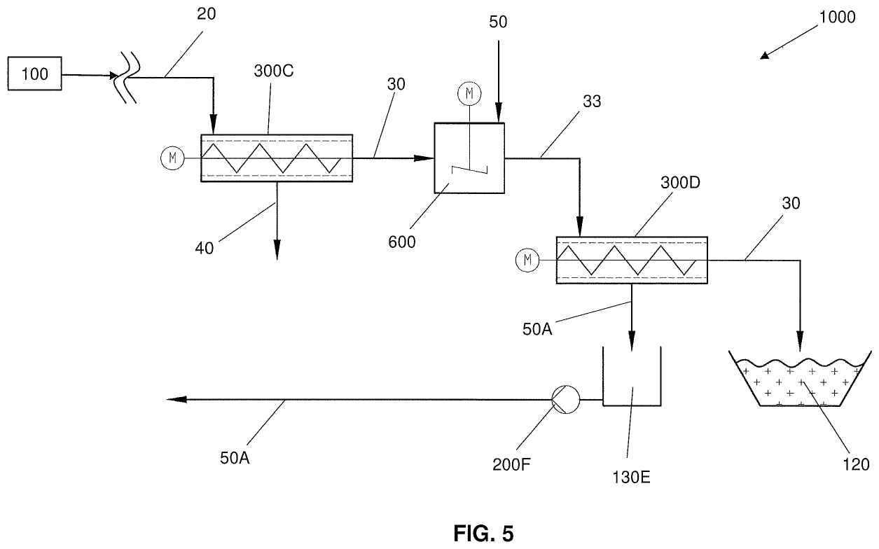

FIG. 5 shows a schematic detail view of a post-treatment stage,

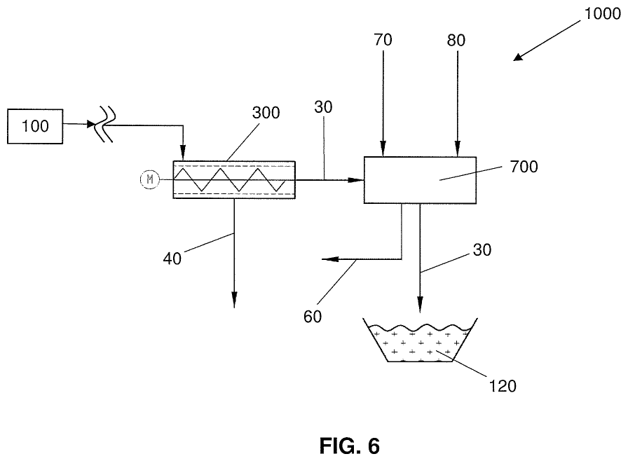

FIG. 6 shows a schematic view of a further post-treatment stage, and

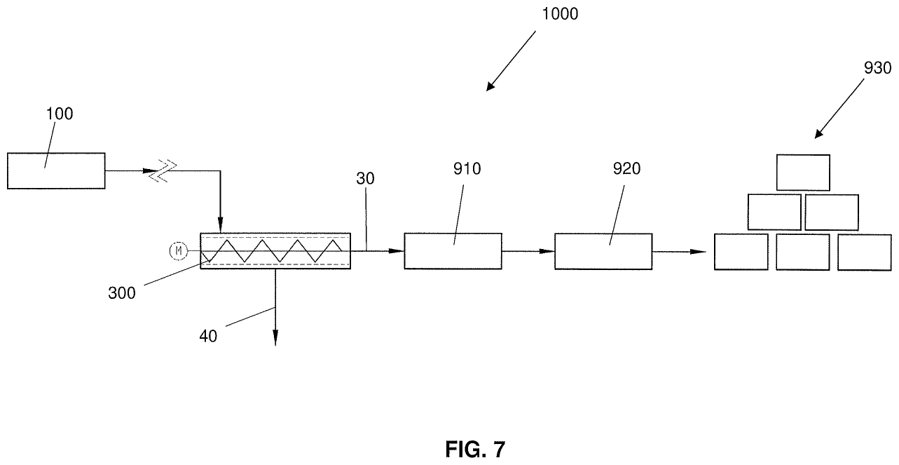

FIG. 7 shows a schematic illustration of a packaging plant.

FIG. 1A schematically shows a first embodiment variant of the plant 1000 according to the invention. According to the invention, the biomass 10 to be treated, which consists of renewable raw materials or organic residues having a high cellulose fibre content, is introduced into a thermo-pressure hydrolysis plant 100 and subjected to a pressure/temperature pre-treatment, namely a thermo-pressure hydrolysis, preferably with steam explosion. During this, the biomass is pulped, resulting in a fibrous sludge 20 having a dry matter content of 10% to 35%, which is collected in a storage tank 110.

By means of a conveying device 200A, for example a screw conveyor or thick-matter pump, the fibrous sludge 20 is introduced into a separation plant 300, typically a screw press, and the fibrous sludge 20 is dewatered, resulting in a fibre press cake 30 having a dry matter content of more than 20%, which is ejected into a collection tank 120. This fibrous solid 30 may either be immediately delivered for further processing, for example to a paper mill, or else it may be subjected to further processing (as described below).

The filtrate 40 from the separation plant 300 is a flowable, high-solids, thin sludge which is collected in an intermediate tank 130 and is subsequently transferred to a biogas plant 2000 as a fermentation substrate by means of a pump device 200B.

In order to improve the separation effect in the separation plant 300, it is preferably provided that filtrate 40 in the form of thin sludge from the intermediate tank 130 is fed to the fibrous sludge 20 from the storage tank 110 via a recirculation line containing a pump device 200C. As an alternative or in addition to this, fresh water 50 or else a filtrate of the thin sludge that is obtained via a separate separation process (not shown) may be fed to the fibrous sludge 20 via a further feed line. By feeding-in liquid, this helps to flush out fines during the separation. At the same time, if recycled filtrate 40 is used, this concentrates the thin sludge, which is ultimately made available to the biogas plant 2000 as a fermentation substrate.

FIG. 1B shows a variant of the plant from FIG. 1A, in which the filtrate 40 from the separation plant 300 is additionally concentrated. The reference signs used in FIGS. 1B and 1n the subsequent figures refer to the same elements of the plant as those already used in FIG. 1A.

In this plant 1000, the thin sludge 40 is channeled from the intermediate storage tank 130 into a filtration unit 800, wherein this filtration unit 800 is designed as a single-stage or multi-stage fine filtration, microfiltration or ultrafiltration plant or combinations thereof. The thickened liquid phase 40B obtained from the filtration unit 800 is fed to the biogas plant 2000 as a fermentation substrate, while the lower-solids filtrate 40A is returned to the intermediate storage tank 130. In this embodiment of the plant 1000, this filtrate can then, if required, be made available again in the process as mashing water, in particular for the fibrous sludge 20 obtained from the thermo-pressure hydrolysis plant 100.

In the variant of the plant 1000 according to the invention that is shown in FIG. 1C, a dispersing of the fibrous sludge 20 in a dispersing unit 900 takes place prior to the separation step in the separation plant 300. This dispersing step takes place at temperatures ≥60° C. with a high energy input by way of a mixing device arranged in the dispersing unit 900, in order to obtain a more even distribution of the fibres in the fibrous sludge 20. It may also be provided here that liquid, preferably recirculated liquid, is added for the sake of better dispersion. This dispersion further improves the subsequent separation of the fibrous sludge 20 into fibre cake 30 and filtrate 40 in the separation plant 300.

FIG. 2A shows a further embodiment variant of the plant 1000 according to the invention, wherein in a first step, as already described in FIGS. 1A and 1B, the biomass 10 is pulped in the thermo-pressure hydrolysis plant 100. The fibre cake 30 obtained from the separation plant 300A and already partially cleaned of fines is fed to a mashing tank 400 (also called a “pulper”) via a feed line, optionally by means of a conveying device, such as for example a screw conveyor, conveyor belt or pump. In the mashing tank, this fibre cake 30 is mixed with recirculated filtrate 41 or alternatively with supplied fresh water 50, or mashing water 60, in order to obtain a dry matter content of usually between 3% and 15% which is favourable for the further treatment of the fibre cake 30. A filtrate of the thin sludge (not shown), which is obtained via a separate separation process, may also be fed in as mashing water. Any foreign materials (for example stones) contained in the raw material sink to the bottom of the mashing tank 400 and can easily be discharged through the bottom outlet 401.

The mashing tank 400 is emptied by means of a further centrifugal pump 200D, which is preferably especially suitable for fibrous media, and the fibre cake 31, to which water has been fed, is routed to a fibre disintegrator 500 (for example a “refiner” or “de-flaker”). In this device 500, the filter cake is exposed to high shear forces by device internals in the form of rotating and static elements.

By means of a de-flaker or refiner, the fibres that are still in the form of bundles are separated, without significantly shortening the fibres themselves. This fibre processing procedure in the form of fibre singulation is also a method step that is necessary in papermaking, this step usually being carried out in the paper mill itself.

As an alternative or in addition to this, the use of a device for the purpose of fibre shortening, in particular a refiner, may also be provided, depending on the biomass 10 used and the desired end product.

Depending on the raw material used, it may be necessary to carry out the fibre singulation and/or fibre shortening in multiple stages. To this end, in the plant 1000 shown in FIG. 2A, the fibrous material 32 obtained in the disintegrator 500 is returned to the mashing tank 400, thereby enabling the fibrous material 32 to pass through multiple times. Fibrous sludge 31 that has not yet been processed may also be fed to the mashing tank 400, as well as, if required, fresh water 50, mashing water 60 and/or recirculated filtrate 41, and added to the fibres 32 that have already been processed in the disintegrator 500. The singulated fibre material is thus optionally fed to the pulper 400 and then to the disintegrator 500 multiple times in a cyclic process. This results in fibres that are better able to be used, and bothersome fines are also separated from the fibres in addition. This thus also increases the fibre purity in the end product. As soon as the fibres are of the quality that is to be achieved in this step, they are fed to a storage tank 140. Alternatively, it may also be provided that the fibres are fed directly to a second separation step, without intermediate storage in the storage tank 140.

In the plant 1000 shown in FIG. 2A, this second separation stage is provided by a further mechanical separation plant 300B, typically a screw press. In this variant of the invention, the fibrous material 32 obtained from the disintegrator 500 is introduced into this second separation plant 300B from the storage tank 140 by means of a conveying device 200E, and the fibres 32 are dewatered to a dry matter content of at least 25%, preferably more than 40%. Water 50 may optionally be introduced into the pressing process in a targeted manner via a feed line. A washing of the press cake 30 optionally additionally takes place, in particular also in the form of a zoned dewatering process. In this way, relatively large quantities of filtrate 41 in the form of thin sludge are again obtained, which are collected in a storage tank 130B.

The filtrate 41 may optionally be reintroduced from the storage tank 130B into the mashing tank 400 via the recirculation line. A feed line for feeding the filtrate 41 into the biogas plant 2000 is also provided.

The plant 1000 shown in FIG. 2B comprises all the plant elements of the plant 1000 from FIG. 2A, with two filtration units 800A, 800B being provided in addition, which respectively process the thin sludge fractions 40, 41 from the two separation plants 300A, 300B. The resulting low-solids filtrates 40A, 41A are returned to the process, preferably added to the fibrous sludge 20 from the thermo-pressure hydrolysis plant 100 and/or added to the mashing tank 400 as mashing water. The high-solids fractions 40B, 41B from the filtration units 800 are again made available to the biogas plant 2000 as a fermentation substrate.

In a further space-saving variant of the plant 1000 according to the invention, as shown in FIG. 3A, again only a single-stage separation process is provided by means of the separation plant 300 which, in contrast to the plant 1000 described in FIGS. 2A and 2B, is arranged downstream of the disintegrator 500, while a separation stage upstream of the pulper 400 has been omitted. In this embodiment of the plant 1000 according to the invention, therefore, after the thermo-pressure hydrolysis of the biomass 10 in the thermo-pressure hydrolysis plant 100, the fibre bundles are immediately singulated in the disintegrator 300 after setting the required (lower) dry matter content in the pulper 400, without further pre-treatment steps.

For this purpose, in a further embodiment of this plant shown in FIG. 3B, at least one filtration unit 800 may again be provided, in which the thin sludge 40 from the separation unit 300 is thickened before being fed to the biogas plant 2000 as a fermentation substrate 40B, while the filtrate 40A is returned to the intermediate storage tank 130.

FIG. 4A shows, in a detail view of a further embodiment of the plant 1000 according to the invention, a variant of the separation stage comprising the separation plant 300, in which the filtrate 40 is collected not in a single storage tank 130, but rather in sub-streams 40C, 40D. In this case, a first sub-stream 40C from at least one first area of the separator 300, which has a higher solids content, is routed to a first storage tank 130C via one outlet line, while a second sub-stream 40D from at least one second dewatering zone of the separator 300, which contains a high proportion of the pressing water stream and thus has a lower solids content, is fed to a second storage tank 130D via a second outlet line.

Preferably, the high-solids filtrate 40C collected in the first storage tank 130C is fed to the biogas plant 2000, while the low-solids filtrate 40D from the second storage tank 130D is fed back via the recirculation line to the pulper 400 for the mashing process. It will be understood that this variant can be used for any separation unit in the plant 1000 according to the invention.

In this connection, it is additionally pointed out that the at least one separation plant 300 may have more than just two different dewatering zones, depending on the way in which it is built and designed. The important thing in this variant of the plant 1000 according to the invention is that at least two sub-streams of filtrate 40C, 40D having a different solids content are collected from the at least one separation plant 300 separately from each other and put to further use.

In one variant of this plant 1000, as shown in FIG. 4B, a filtration unit 800 may be provided, which further concentrates the higher-solids fraction 40C from the separation plant 300. The high-solids fraction 40E from the filtration plant 800 is in this case fed to the biogas plant 2000, while the lower-solids filtrate 40F from the filtration unit 800 is routed into the intermediate storage tank 130D and, if required, is routed jointly with the sub-fraction 40C from the separation plant 300 into the process as process water, for example for mashing purposes.

In the further variant of the plant 1000 according to the invention that is shown in a detail view in FIG. 5, a further treatment stage comprising a mixing reactor 600 is provided downstream of a separation plant 300C. In this mixing reactor 600, the fibrous material 30 obtained from the separation plant 300C is mixed with wash water 50 that is fed in via a feed line. The contaminated wash water 50A from the mixing reactor 600 is separated from the cleaned fibrous material 33 in a further separation plant 300D, and the end product 30 is fed to the collection tank 120.

In an alternative embodiment, it is provided that the mixing reactor 600 and the separation plant 300D are designed as a structural unit, for example in the form of a washing drum having a compression zone, or integrated in a screw conveyor having a pressing and dewatering zone.

The filtrate 50A thus produced is collected in a storage tank 130E and, if required, is fed to the thermo-pressure hydrolysis plant 100 and/or to the mashing tank 400 by means of a pump device 200F, for example as mashing water, in order to adjust the raw material located therein to a suitable water content.

Of course, this additional treatment stage can additionally or alternatively be used in any of the aforementioned plant variants shown in FIGS. 1A to 4B in combination with the respective separation plants 300, 300A, 300B.

FIG. 6 shows an optional post-treatment of the pulp produced in the method according to the invention. For this, the pulp 30 obtained from the separation plant 300 is stabilized in a post-treatment reactor 700 by means of conditioning chemicals 70 and process heat 80. Of course, it may also be provided that the post-treatment takes place only by means of conditioning chemicals, or exclusively by a heat treatment. In addition or as an alternative, the pulp may additionally be dried in a suitable device, in particular in the post-treatment reactor 700, wherein it is particularly preferably provided that this heat treatment takes place using process heat 80 from the biogas plant 2000 and/or from the thermo-pressure hydrolysis plant 100. This use of waste heat also has a positive effect on the energy balance of the method according to the invention.

The condensates and/or effluent occurring in the post-treatment may be returned to the post-treatment and/or may also be used as process water.

FIG. 7 schematically shows an optional compacting and packaging of the pulp 30 produced in the method according to the invention. For this, the pulp 30 obtained from the at least one separation plant 300 (with or without post-treatment) is compacted in a high-pressure press 910 to form cuboid or cylindrical bales, and the bales thus produced are wrapped with a film or another suitable fabric in a packaging plant 920 in order in this way to obtain storable, easy-to-handle bales, which can then be safely stored and transported in the form of bale stacks 930.

The method according to the invention using the associated plants may in principle be operated as a continuous system or as a cyclic system. Mixed operation is also conceivable, in which, for example, the separation plants are operated continuously, while the mashing and/or disintegrating steps take place intermittently.

1. A method for obtaining cellulose fibres from fibrous biomass, comprising:
subjecting the biomass to thermo-pressure hydrolysis, preferably with steam explosion, in a thermo-pressure hydrolysis plant, and
separating the fibrous sludge obtained from the thermo-pressure hydrolysis plant in at least one separation plant, wherein:
a press cake formed of cellulose fibres, preferably having a dry matter content of more than 20%, preferably more than 25%, and a filtrate formed of a flowable, high-solids, thin sludge are obtained, and
the thin sludge is fed to a biogas plant as a fermentation substrate in order to obtain biogas.

2. The method according to claim 1, wherein the fibrous sludge obtained after the thermo-pressure hydrolysis is dispersed in a dispersing unit, preferably at a temperature T≥60 C.

3. The method according to claim 1, wherein:
the fibrous sludge obtained after the thermo-pressure hydrolysis is adjusted in a subsequent step to a dry matter content of preferably 3% to 20%, particularly preferably 3% to 10%, and
the adjustment preferably takes place in a mashing tank, and then the separation of the mashed fibrous sludge takes place in at least one separation plant.

4. The method according to claim 3, wherein, before the separation in the at least one separation plant, first a fibre separation and/or fibre shredding of fibre bundles in the mashed fibrous sludge from the mashing tank takes place in at least one disintegrator, and then a separation of the fibrous sludge takes place in the at least one separation plant.

5. The method according to claim 1, wherein, after the fibrous sludge from the thermo-pressure hydrolysis plant has been separated in at least one first separation plant:
the press cake obtained is fed to a mashing tank in order to set a dry matter content of preferably 3% to 20%, particularly preferably 3% to 10%,
the mashed fibrous sludge is fed to at least one disintegrator in order to obtain a fibre separation and/or fibre shredding of the fibre bundles contained in the mashed fibrous sludge, and
a separation of the fibrous sludge takes place in at least one second separation plant.

6. The method according to claim 4, wherein:
the fibre separation and/or fibre shredding in the at least one disintegrator is repeated at least once, preferably multiple times, and
the fibrous sludge is routed between the mashing tank and the disintegrator preferably in a cyclic process.

7. The method according to claim 1, wherein the filtrate from the at least one separation plant, which is in the form of a thin sludge, is at least in part fed to the biogas plant as a fermentation substrate.

8. The method according to claim 7, wherein the filtrate from the at least one separation plant, which is in the form of a thin sludge, is collected and is at least in part returned to the process, in particular is fed to the mashing tank and/or is added to the fibrous sludge upstream of the at least one separation plant.

9. The method according to claim 7, wherein:
the thin sludge is collected in two sub-fractions, and
a first sub-fraction having a lower solids content is returned to the process, while a higher-solids fraction is fed to the biogas plant as a fermentation substrate.

10. The method according to claim 1, wherein the press cake obtained from the at least one separation plant is subjected to a stabilization step, in particular by adding preserving chemicals, and/or to a heat treatment, preferably by supplying process heat.

11. The method according to claim 1, wherein:
the press cake obtained from the at least one separation plant is subjected to a further cleaning step in a mixing reactor, and
the wash water is separated from the cleaned fibre cake in a further separation plant.

12. The method according to claim 11, wherein the wash water is collected and is preferably fed to the biomass upstream of or in the thermo-pressure hydrolysis plant in order to adjust the water content.

13. The method according to claim 1, wherein the press cake obtained from the at least one separation plant is compacted and then packaged in order to obtain storable, easy-to-handle bales.

14. The method according to claim 1, wherein:
the filtrate from the at least one separation plant, which is in the form of a thin sludge, is separated in a further processing step, in particular in a filtration device, into a high-solids, thickened thick phase and into a low-solids filtrate, and
the thick phase is made available to the biogas plant as a fermentation substrate, while the filtrate is returned to the process, in particular as dilution water or mashing water.

15. The method according to claim 1, wherein the fibrous biomass used is plant biomass, in particular energy crops such as maize, Silphium perfoliatum, and/or harvest residues having a sufficient cellulose or lignocellulose content, such as straw and/or green cuttings.

16. The method according to claim 1, wherein the use of the biogas obtained in the biogas plant as an energy source, and/or of the waste heat from the biogas plant, in particular for the thermo-pressure hydrolysis plant.

17. The method according to claim 1, wherein the use of the non-recyclable residues occurring in the biogas plant, in particular containing lignin and/or silicates, as fertilizing and soil improvement agents in agriculture.

18. A plant for carrying out the method a method according to claim 1, for obtaining cellulose fibres from fibrous biomass, the plant comprising:
at least one thermo-pressure hydrolysis plant for subjecting the fibres of the biomass firstly to thermo-pressure hydrolysis, preferably with steam explosion, wherein:
the thermo-pressure hydrolysis plant is connected via at least one feed line to at least one separation plant, preferably a screw press, into which the fibrous sludge drawn off from the thermo-pressure hydrolysis plant can be fed by means of at least one conveying device, preferably a screw conveyor and/or a thick-matter pump, and
the filtrate obtained from the at least one separation plant in the form of a flowable, high-solids, thin sludge can be fed to a biogas plant via at least one further feed line.

19. The plant according to claim 18, further comprising:
at least one dispersing unit, which is arranged between the thermo-pressure hydrolysis plant and the at least one separation plant.

20. The plant according to claim 18, further comprising:
a mashing tank, which is arranged between the thermo-pressure hydrolysis plant and the at least one separation plant.

21. The plant according to claim 20, wherein:
the at least one mashing tank is connected to at least one disintegrator, and
the at least one disintegrator is connected to the at least one separation plant preferably via at least one storage tank.

22. The plant according to claim 20, wherein:
the at least one mashing tank is arranged downstream of the at least one separation plant,
the mashing tank is connected to the at least one disintegrator, and
the at least one disintegrator is connected to at least one further separation plant preferably via at least one storage tank.

23. The plant according to claim 18, wherein the filtrate from the at least one separation plant can be collected in at least one collection tank.

24. The plant according to claim 23, wherein the at least one collection tank is connected to the mashing tank via at least one recirculation line and/or to the biogas plant via at least one further feed line.

25. The plant according to claim 18, wherein:
the at least one separation plant is connected to at least one further cleaning device for carrying out an additional cleaning step in order to clean the press cake obtained from the at least one separation plant, and
the cleaning device is preferably designed as a mixing reactor with at least one further separation plant.

26. The plant according to claim 25, wherein the mixing reactor with the at least one further separation plant is designed as a structural unit, preferably as a washing drum having a compression zone or as a screw conveyor having a pressing and dewatering zone.

27. The plant according to claim 18 further comprising:
at least one filtration unit is provided, in which at least one filtrate from the at least one separation plant can be separated into a high-solids, thickened thick phase and into a low-solids filtrate, wherein:
the thick phase is made available to the biogas plant as a fermentation substrate, while the filtrate can be returned to the process, in particular as dilution water or mashing water, via at least one recirculation line.
